(19) United States
(12) Patent Application Publication
(10) Pub. No.: US 2025/0149126 A1
(43) Pub. Date: May 8, 2025

(54) DIGITAL FRAMEWORK FOR DESIGN AND SELECTION OF PARAFFIN INHIBITORS

(71) Applicant: Schlumberger Technology Corporation, Sugar Land, TX (US)

(72) Inventors: Bastian Sauerer, Houston, TX (US); Moein Zeynolabedinpour, Houston, TX (US); Kang Wang, Houston, TX (US); Chandrashekhar Khandekar, Humble, TX (US)

(21) Appl. No.: 18/934,581

(22) Filed: Nov. 1, 2024

Related U.S. Application Data
(60) Provisional application No. 63/596,013, filed on Nov. 3, 2023.

(51) Int. Cl. G16C 20/30; G01N 33/28; G16C 20/70; G16C 20/90

(52) U.S. Cl. CPC G16C 20/30; G01N 33/2823; G16C 20/70; G16C 20/90

(57) ABSTRACT

A method of selecting paraffin inhibitors for a target crude oil includes inputting one of more known properties of a target crude oil into a machine learning model and extrapolating unknown properties of a historical data set using a machine learning model. The machine learning model is trained on the historical data set that includes one or more properties of a plurality of crude oils, one or more properties of a plurality of paraffin inhibitors, and one or more paraffin inhibiting efficiencies of the paraffin inhibitors with the plurality of crude oils. Paraffin inhibiting efficiency is predicted based on the historical data set and extrapolated unknown properties for the plurality of paraffin inhibitors that may be used with the target crude oil. One or more of a list of crude oils having one or more properties within a numerical tolerance of the properties of the target crude oil, a list of paraffin inhibitors for use with the target crude oil, and a list of paraffin inhibitor properties are output.

### CROSS-REFERENCE TO RELATED APPLICATIONS

The present application claims priority to and the benefit of U.S. Provisional Patent Application Ser. No. 63/596,013 titled “A DIGITAL FRAMEWORK FOR DESIGN AND SELECTION OF PARAFFIN INHIBITORS” filed Nov. 3, 2023, the disclosure of which is incorporated herein by reference in its entirety.

### BACKGROUND OF THE DISCLOSURE

Crude oil is comprised primarily of hydrocarbon fluids. During oil production, crude oil is transported from a geological formation to the surface of the earth through production tubing and wellbore equipment. Once on the surface, the crude oil is processed and transported through pipelines. Such hydrocarbon fluids can contain a class of high molecular weight molecules, often referred to as paraffin, wax, or paraffin wax, that under certain circumstances can precipitate and deposit within the piping or equipment used to transport and process the hydrocarbon fluids. These paraffins are generally alkane molecules. Growth of such deposits can partially or completely plug the flow paths through the piping and equipment; slowing or stopping hydrocarbon processing until the deposits can be cleared.

To minimize or slow the formation of paraffin deposits, paraffin inhibitors may be added to the crude oil. However, the properties of crude oil can be dramatically different between each source and geological formation. The difference in crude oil properties affects the effectiveness of each type of paraffin inhibitor with some being highly effective at certain concentrations and others having negligible effect on the formation and growth of paraffin deposits. In addition, testing can be expensive and time consuming to identify which paraffin inhibitors may be effective with a certain crude oil.

### SUMMARY

In some aspects, the techniques described herein relate to a method for selecting paraffin inhibitors for a target crude oil. The method includes inputting one or more known properties of a target crude oil into a machine learning model. A machine learning model extrapolates unknown properties of a historical data set. The machine learning model is trained on the historical data set including one or more properties of a plurality of crude oils, one or more properties of a plurality of paraffin inhibitors, and one or more paraffin inhibiting efficiencies of the paraffin inhibitors with the plurality of crude oils. The machine learning model predicts a paraffin inhibiting efficiency based on the historical data set and extrapolated unknown properties for the plurality of paraffin inhibitors that may be used with the target crude oil and outputs one or more of: a list of crude oils having one or more properties within a numerical tolerance of the properties of the target crude oil, a list of paraffin inhibitors for use with the target crude oil, and a list of paraffin inhibitor properties.

In some aspects, the techniques described herein relate to a method that includes adding, to a database, a plurality of records of crude oils and paraffin inhibitors. Each record includes one or more of: properties of the crude oils including identification of a source of a crude oil, paraffin content, carbon chain length distribution of paraffins, wax appearance temperature, American Petroleum Institute (API) gravity, cloud point, pour point, and a ratio of normal paraffins to branched plus cyclic paraffins, and saturates, aromatics, resins, asphaltenes (SARA) fractions, identification of a paraffin inhibitor, type of the paraffin inhibitor, molecular properties of the paraffin inhibitor, dosage of the paraffin inhibitor in the crude oil, and paraffin inhibition results of the paraffin inhibitor in the crude oil. The method includes generating first model parameters to compare a target crude oil to the crude oils in the database and generates second model parameters to compute a paraffin inhibitor efficiency of the paraffin inhibitors in the database with the target crude oil. The method includes generating third model parameters to compute a confidence level of the paraffin inhibitor efficiency of the paraffin inhibitors in the database with the target crude oil. The method includes inputting properties of the target crude oil into a model containing the first model parameters, the second model parameters, and the third model parameters and outputting one or more of: a list of crude oils having one or more properties within a numerical tolerance of the properties of the target crude oil, a list of paraffin inhibitors for the target crude oil, each including a paraffin inhibitor efficiency, and a list of inhibitor properties.

In some aspects, the techniques described herein relate to a computing system that includes: a processor and a memory including instructions, a database, and a machine learning model. The machine learning model causes the processor to receive one of more known properties of a target crude oil into the machine learning model and extrapolate unknown properties of a historical data set using the machine learning model. The machine learning model is trained on the historical data set including one or more properties of a plurality of crude oils, one or more properties of a plurality of paraffin inhibitors, and paraffin inhibiting efficiencies of the paraffin inhibitors with the crude oils. The machine learning model causes the processor to predict a paraffin inhibiting efficiency for the one or more paraffin inhibitors used with the target crude oil based on the extrapolated unknown properties and output one or more of: a list of crude oils having one or more properties within a numerical tolerance of the properties of the target crude oil, a list of paraffin inhibitors for use with the target crude oil, and a list of inhibitor properties.

This summary is provided to introduce a selection of concepts that are further described in the detailed description. This summary is not intended to identify key or essential features of the claimed subject matter, nor is it intended to be used as an aid in limiting the scope of the claimed subject matter. Additional features and aspects of embodiments of the disclosure will be set forth herein, and in part will be obvious from the description, or may be learned by the practice of such embodiments.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments are described and explained with additional specificity and detail through the use of the accompanying drawings in which:

FIG. 1 is an example of transportation equipment for transporting crude oil, according to at least one embodiment of the present disclosure;

FIG. 2 is a schematic side view of a cold finger test assembly used for paraffin inhibition testing, according to at least one embodiment of the present disclosure;

FIG. 3 is a schematic diagram of a model for identifying and recommending paraffin inhibitors, according to at least one embodiment of the present disclosure;

FIG. 4 is a schematic diagram of a decision tree used by the model for identifying and recommending paraffin inhibitors, according to at least one embodiment of the present disclosure;

FIG. 5 is a user interface for inputting data into the model, according to at least one embodiment of the present disclosure;

FIG. 6 a user interface displaying output data from the model, according to at least one embodiment of the present disclosure

FIG. 7 is an example of exported data shown in a spreadsheet, according to at least one embodiment of the present disclosure;

FIG. 8 is a schematic representation of the system, according to at least one embodiment of the present disclosure;

FIG. 9 illustrates certain components that may be included within a computer system, according to at least one embodiment of the present disclosure;

FIG. 10 is a flow chart of a method for selecting paraffin inhibitors for a target crude oil, according to at least one embodiment of the present disclosure;

FIG. 11 is a flow chart of another method according to one embodiment, according to at least one embodiment of the present disclosure;

FIG. 12 is flow chart of yet another method, according to at least one embodiment of the present disclosure.

### DETAILED DESCRIPTION

This disclosure generally relates to devices, systems, and methods for identifying and recommending paraffin inhibitors that may have a high paraffin inhibitor efficiency when used with a target crude oil. Problems that the methods of this disclosure seek to overcome include limited historical data for the paraffin inhibitors that have been tested with the crude oils. Further, the historical data may contain data for tests that may not follow a standardized procedure. Consequently, a method is needed that can extrapolate the historical data into a form that allows reliable predictions to be made about the interactions between crude oils and different paraffin inhibitors, as well as identify correlations between the properties of crude oils and the properties of paraffin inhibitors.

A method for selecting paraffin inhibitors that have a high paraffin inhibitor efficiency for a target crude oil may include a user inputting one of more known properties of a target crude oil into a machine learning model that has been trained by a historical data set that includes one or more properties of a plurality of crude oils, one or more properties of a plurality of paraffin inhibitors, and one or more paraffin inhibiting efficiencies of the paraffin inhibitors with the crude oils. Historical data also may include testing conditions of paraffin inhibitors with crude oils such as cold finger temperature, surface roughness, coefficient of friction, and materials of the testing apparatus, bath temperature, time segments of the testing procedures, and set-up and take down procedures of each test. The historical data set includes cold finger test results of a combination of a crude oil and a paraffin inhibitor. The machine learning model extrapolates unknown properties of the target crude oil and data sets within the historical data set, such as connections or relationships between the crude oil and properties of the paraffin inhibitor. The machine learning model predicts a paraffin inhibiting efficiency based on the historical data set and extrapolated unknown properties for the one or more paraffin inhibitors that may be used with the target crude oil. The machine learning model outputs one or more of a list of crude oils having one or more properties within a numerical tolerance of the properties of the target crude oil, a list of paraffin inhibitors for use with the target crude oil; and a list of paraffin inhibitor properties.

A user or the machine learning model may select or design a chosen paraffin inhibitor, based on the output, for use with the target crude oil. In one embodiment, the user may test a plurality of the output paraffin inhibitors with the target crude oil. Based on the actual test data, the user selects the paraffin inhibitor to be used with the target crude oil. Once selected, a user may validate the predicted paraffin inhibiting efficiency with testing on the paraffin inhibitor and the target crude oil, and add the results of the actual testing to the historical data set.

The one or more properties of a plurality of crude oils may include a location of a source of one of the plurality of crude oils. The location of a source of a crude oil may allow the machine learning model to identify additional data sets within the historical data that may be closely related to the crude oil and facilitate the identification of effective paraffin inhibitors. The one or more properties of a plurality of crude oils may also include carbon chain distribution of the paraffins, or the wax appearance temperature (WAT). Both of these properties help the machine learning model identify the type and molecular properties of a paraffin inhibitor that may be effective with a particular crude oil.

The one or more properties of a plurality of paraffin inhibitors may include one or more of polymer length, side chain length, or the ratio between polymer length and side chain length. Similarly, the molecular properties of paraffin inhibitors may facilitate the machine learning model in identifying and building correlation models to improve its ability to predict the structure and composition of an effective paraffin inhibitor with a specific crude oil.

In some embodiments, the machine learning model may design a paraffin inhibitor and include the newly designed paraffin inhibitor as an output. Without actual test data for the newly designed paraffin, a user will test the newly designed paraffin inhibitor with the target crude oil. Once tested, a user will add the results of the actual testing to the historical data set. This data set for the newly designed paraffin may be used to calibrate the machine learning model.

As part of the method, the machine learning model may calculate a paraffin inhibitor efficiency value for each combination of a paraffin inhibitor and the target crude oil using the historical data set. Prior to the outputting step, the machine learning model may order the paraffin inhibitors for use with the target crude oil based on the predicted paraffin inhibitor efficiency value. As used herein, the term “ordering” means to arrange in a methodical way and should be interpreted to include the ranking of paraffin inhibitors by paraffin inhibitor efficiency. For example, ordering the paraffin inhibitors may mean placing the most efficient paraffin inhibitor at the top of an outputted list. Alternatively, ordering the paraffin inhibitors may mean merely indicating a set of paraffin inhibitors have good efficiency compared to other paraffin inhibitors that have poor efficiency with a target crude oil.

The machine learning model may calculate a confidence level for the paraffin inhibitor efficiency using a confidence indicator based on one or more of a numerical algorithm, statistical calculation, a decision tree classifying one or more properties of a plurality of crude oils, one or more properties of a plurality of paraffin inhibitors, or a cold finger test result in the historical data set. The machine learning model may use the confidence level to limit the output to one or more crude oils, paraffin inhibitors, and inhibitor properties that have a confidence level above a predetermined threshold.

The machine learning model in predicting the paraffin inhibiting efficiency may use a Bayesian Network to relate the paraffin inhibitor efficiency to the range of values representing the crude oil properties and paraffin inhibitor structural data.

Another method of the disclosure may have a user or a machine learning model add, to a historical database, a plurality of records of crude oils and paraffin inhibitors. Each record includes one or more of properties of the crude oil including identification of a source of a crude oil, paraffin content, carbon chain length distribution of paraffins, wax appearance temperature, American Petroleum Institute (API) gravity, cloud point, pour point, and a ratio of normal paraffins to branched plus cyclic paraffins, and saturates, aromatics, resins, and asphaltenes (SARA) fractions, identification of a paraffin inhibitor, type of the paraffin inhibitor, molecular properties of the paraffin inhibitor, dosage of the paraffin inhibitor in the crude oil and paraffin inhibition results of the paraffin inhibitor in the crude oil. The machine learning model generates first model parameters to compare a target crude oil to the crude oils in the database. The machine learning model generates second model parameters to compute a paraffin inhibitor efficiency of the paraffin inhibitors in the database with the target crude oil and generates third model parameters to compute a confidence level of the paraffin inhibitor efficiency of the paraffin inhibitors in the database with the target crude oil. The machine learning model inputs properties of the target crude oil into a model containing the first model parameters, the second model parameters, and the third model parameters. The machine learning model then outputs one or more of a list of crude oils having one or more properties within a numerical tolerance of the properties of the target crude oil, a list of paraffin inhibitors for the target crude oil, each including a paraffin inhibitor efficiency, and a list of inhibitor properties.

The machine learning model or a user may select or design a chosen paraffin inhibitor, based on the output, for use with the target crude oil. In one embodiment, the user may test a plurality of the output paraffin inhibitors with the target crude oil. Based on the actual test data, the user selects the paraffin inhibitor to be used with the target crude oil. Once selected, a user may validate the confidence level with testing on the paraffin inhibitor and the target crude oil and add the results of the actual testing to the historical data set. The historical data set may include a cold finger test results of a combination of a crude oil and a paraffin inhibitor.

The machine learning model may extrapolate unknown properties of each record using the database to generate one or more of the first model parameters, second model parameters, and third model parameters. The machine learning model may predict the paraffin inhibiting efficiency using a Bayesian network to relate the paraffin inhibitor efficiency to the range of values representing the crude oil properties and paraffin inhibitor structural data.

In computing a confidence level for the paraffin inhibitor efficiency, a machine learning model may use a confidence indicator based on but not limited to statistical models, numerical algorithms, or a decision tree classifying one or more properties of a plurality of crude oils, one or more properties of a plurality of paraffin inhibitors, or a cold finger test result in the database. The machine learning model may use the confidence level to limit the output to one or more crude oils, paraffin inhibitors, and inhibitor properties that have a confidence level above a predetermined threshold.

A computing system includes a processor and a memory including instructions, a database, and a machine learning model that may cause the processor to implement the method. The computer system receives one of more known properties of a target crude oil into the machine learning model. The computer system then extrapolates unknown properties of a historical data set using the machine learning model. The machine learning model is trained on the historical data set including one or more properties of a plurality of crude oils, one or more properties of a plurality of paraffin inhibitors, and paraffin inhibiting effectiveness of the paraffin inhibitors with the crude oils. The computer system predicts a paraffin inhibiting efficiency for the one or more paraffin inhibitors used with the target crude oil based on the extrapolated unknown properties. The computer system then outputs one or more of a list of crude oils having one or more properties within a numerical tolerance of the properties of the target crude oil, a list of paraffin inhibitors for use with the target crude oil, and a list of inhibitor properties.

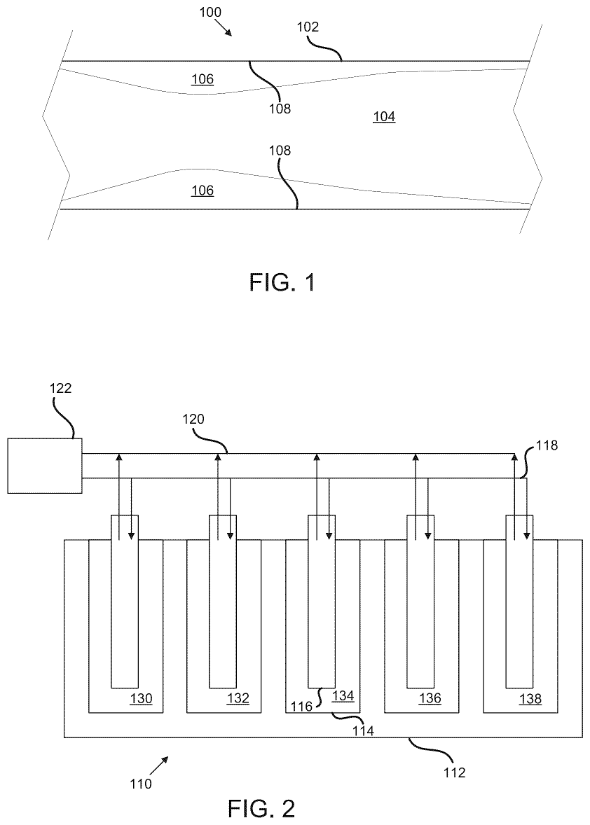

FIG. 1 shows an example of equipment 100 for transporting crude oil. Equipment 100 may be any enclosure that holds or moves crude oil within it, including but not limited to piping used in extraction from oil wells, pipelines, holding tanks, and pipes within crude oil processing equipment. The equipment 100 may be a pipe 102 transporting crude oil 104. During transport and storage, long chain hydrocarbons including paraffins 106 may precipitate out of the crude oil and deposit on the interior walls 108 of the pipe 102. In order to slow the deposition of paraffins 106 on the interior walls 108 of the pipe 102, paraffin inhibitors may be added to the crude oil 104. As used herein, the term paraffin inhibitor may include paraffin dispersants and pour point depressants in addition to paraffin inhibitors.

FIG. 2 shows a schematic side view of a cold finger test assembly 110. The ability of a specific paraffin inhibitor to inhibit the deposition of paraffins on the interior walls 108 of the pipe 102 (shown in FIG. 1) can be quantified using a cold finger test assembly 110. The cold finger test assembly 110 includes a hot block 112 used to warm crude oil samples 130, 132, 134, 136, 138. The cold finger test assembly 110 may be configured to test more or fewer samples than shown. A wide array of sensors (not shown) may be used to measure and control the test parameters of the cold finger test assembly 110. In addition, each crude oil may be thoroughly analyzed to determine the properties of each crude oil and recorded with each cold finger test. Similarly, each paraffin inhibitor may be thoroughly analyzed to determine the properties of each paraffin inhibitor and recorded with each cold finger test.

The crude oil samples 130, 132, 134, 136, 138 may be containerized (not shown) and are disposed in a plurality of wells 114 formed in the hot block 112. The oil samples 130, 132, 134, 136, 138 are typically stirred during testing to simulate movement with a pipe. The hot block 112 may be heated using electrical resistance heater, hot fluid, or any other way that can be used to maintain the hot block 112 at a desired temperature or change the temperature as desired. A finger 116 is disposed within each well 114. The fingers 116 may be made of metal or ceramic. Refrigerant is circulated to an interior of each finger 116 through a refrigerant inlet 118 and outlet 120. A chiller 122 maintains temperature of the refrigerant in the inlet 118. To perform a test, the fluid within the sample vials is maintained at a temperature above the wax appearance temperature of the crude oil, while the cold fingers are maintained at a temperature lower than the wax appearance temperature of the crude oil. After a predetermined duration, the cold fingers 116 are removed. A deposited mass of paraffin can be ascertained with and without different paraffin inhibitors and concentrations of paraffin inhibitor in the oil samples 130, 132, 134, 136, 138.

A paraffin inhibition efficiency (PIE) may be determined from cold finger testing and is one of the parameters used to identify the effectiveness of a specific paraffin inhibitor with a specific crude oil. The paraffin inhibition efficiency is a function of the crude oil and the paraffin inhibitor, which can be determined by the following equation:

$$P I E = ( ( W o - Wi ) / Wo ) \times 100$$

A weight of a mass of paraffin deposited by one of the oil samples 130, 132, 134, 136, 138 that was treated with a paraffin inhibitor is Wi and a weight of a mass of paraffin deposited from one of the oil samples 130, 132, 134, 136, 138 not treated with a paraffin inhibitor is Wo. The paraffin inhibition efficiency function parameterizes paraffin deposition inhibition in the crude oil using the paraffin inhibitor. The paraffin inhibition efficiency can also be a function of temperature differential between the oil bath and the cold fingers, and the dosage of inhibitor used in the oil. However, when test parameters, such as temperatures, time of test, speed of stirring, size of sample, dosage of inhibitor, etc. are not standardized, the PIE of one test may not be easily comparable with the results of another test. In addition, tests may report only a portion of the gathered test data, further limiting the ability to use and compare test data to determine a suitable paraffin inhibitor for a specific crude oil.

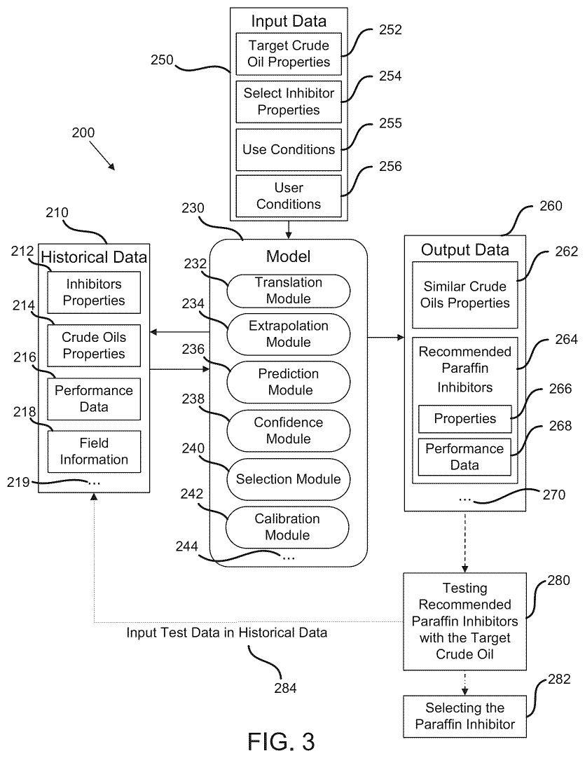

FIG. 3 shows a schematic view of a system 200 to facilitate the selection of a suitable paraffin inhibitor for use with a specific crude oil, according to at least one embodiment of the present disclosure. As shown, paraffin inhibitors properties 212 and the crude oils properties 214 may be part of a historical database or more generally form part of historical data 210.

The paraffin inhibitors properties 212 may include the names of paraffin inhibitors and the polymer chemistry of paraffin inhibitors, including structure type such as comb, star, dendrimer, etc., and structural characteristics of an inhibitor, such as main chain length, functional groups, and number and length of the pendent side chains. Further, paraffin inhibitors properties 212 include the distribution of different chain length polymers and concentrations of additional chemicals in a paraffin inhibitor, including paraffin dissolvers. The paraffin inhibitors properties 212 may also include any noted synergistic effects when mixed with other paraffin inhibitors and paraffin dissolvers.

The crude oils properties 214 may include the name of a crude oil, its source location, a description of the geological formation it was obtained from, and its distribution of hydrocarbon chain length. In some cases, a weight fraction or weight percent is provided for hydrocarbon chain lengths equal to or greater than C17 and is sometimes given as C17+ or C17-C60+. The crude oils properties 214 may include wax appearance temperature and wax disappearance temperature for a crude oil, its American Petroleum Institute (API) gravity, its pour point, cloud point, its ratio of normal paraffins to branched and cyclic paraffins, its weight percentage or weight fraction of saturates, aromatics, resins, and asphaltenes (SARA fractions). Further, the sulfur content and other elemental, chemical, and inert contents may also be included as one of the crude oils properties 214.

Additionally, the historical data 210 may include performance data 216, which includes the parameters, procedures, and results of cold finger tests, including paraffin inhibition efficiency, bath temperature, cold finger temperature, temperature differential, dosage, stirring rate, refrigerant input and output temperature or enthalpy, and composition and weight of the paraffin formation or wax formation. Performance data 216 may also include the mechanical properties of paraffin formations, including paraffin formations of crude oils having paraffin inhibitors mixed in at different dosages. For example, the density, shear strength, tensile strength, modulus of elasticity, hardness, and crystalline structure as a function of temperature of a paraffin formation may be included in the performance data 216. The melting temperature may also be included.

Performance data 216 may include a wide variety of experiment procedures and the resulting test data. Test data indicating changes in a deposited mass of paraffin over time may be included. Unsuccessful test data may also be included in the historical data 210 to provide diverse performance profiles and may allow the model 230 to identify potential areas for tests and data to be gathered.

Historical data 210 may also be included in field information 218, such as in field reports of paraffin build up in equipment 100, journal articles, published information, and the current and historical pricing of paraffin inhibitors, precursor materials, and manufacturing costs. Anticipated field conditions for use of a paraffin inhibitor with a target crude oil may be included to inform selection of test conditions for inclusion in the model 230. Field information 218 may include environmental and field conditions such as crude oil bulk temperature, pipeline outer wall temperature, weather conditions around a pipeline or production location, flow rates, and pipeline and equipment materials and associated surface friction. Field information 218 may also include the thermal flux across a pipeline outer wall. Other information 219 not related to the use of paraffin inhibitors may also be included in the historical data, such as weather reports for specific geographic locations, maps, and geologic formation data.

The historical data 210 may be used to train a model 230. The model 230 uses the historical data 210 to identify potential correlations and similarities in the historical data to make recommendations of potential paraffin inhibitors that may be used with a target crude oil. An existing challenge with the historical data 210 is that it is quite limited in the data available. Additionally, there is great variation between existing test data and testing procedures. This variation in the historical data 210 may limit what can be confidently compared between data sets in the historical data 210 using ordinary means.

The model 230 may be constructed as a model to which machine learning techniques can be applied. In one embodiment, if the model 230 is to be used to select a list of inhibitors that may be used with a target crude oil, the model 230 may be a classification type model. If the model is to be used to predict an effectiveness index for a pairing of a target crude oil with a selected paraffin inhibitor, the model 230 may be a regression type model. The model 230 may include modules that perform specific types of evaluations. For example, one module may perform a classification type evaluation while another module performs a regression type evaluation. For example, one module may select a list of inhibitors likely to be successful for a target crude oil, and another module may predict inhibition efficiency for each of the selected inhibitors with the target crude oil. For example, the model can contain any or all of a KNN algorithm, a random forest algorithm, a Bayesian network algorithm, a response surface algorithm, a fractional factorial algorithm, or other similar algorithms.

As shown, the model 230 may include multiple modules, including a translation module 232, an extrapolation module 234, a prediction module 236, a confidence module 238, a selection module 240, a calibration module 242 and other modules 244. The model 230 may be a machine learning model, a large language model, or use artificial intelligence to train itself on the historical data 210 to identify potential correlations between the paraffin inhibitors properties 212 and the crude oils properties 214 using the performance data 216, and in some embodiments, the field information 218.

The translation module 232 provides a translation function to the model 230. One challenge with the historical data 210 is the use of inconsistent terminology for the same information. The translation module 232 may review the language of the historical data 210 and translate the disparate terminology into a uniform set of terminology including converting the units into a standardized set of units. For example, a data set only providing weight fractions of saturates, aromatics, resins, and asphaltenes may be translated to provide the weight percent of saturates, aromatics, resins, and asphaltenes to facilitate the comparison of data within the historical data 210. Further, the translation module 232 may translate the units of different measurements from metric to imperial values as desired by a user.

The translation module 232 may also translate data in other languages to the language used by a user of the model or as requested by the user. The translation module may be connected to or be able to access a large language model to assist with the translation of historical data into a set of uniform terminology.

The translation module 232 may also include a confidence interval with translated language, unit, molecular description, or source description of historical data 210. In some embodiment, if the confidence interval of a translation falls below a preselected threshold, the translation module 232 may tag the translation to indicate a low confidence level. This may be helpful facilitate human training of the translation module 232.

Different descriptions of the same source location and description of a geological formation can be unified so that sets of data related from differently labeled samples of crude oil may be attributed to the same geological formation or source to increase the data available for the identification and recommendation of paraffin inhibitors with a relatively high likelihood of success for use with a specific crude oil. For example, the translation module 232 may obtain source information for four crude oil samples that are labeled “Midland Basin”, “Wolfberry”, “Ector County”, and “Upton County”, may organize all of these samples and associated data together but then add a confidence level to each sample and associated data based on the source labels and the similarity of the associated data.

As the translation module 232 collates the data within the historical data 210, the improved historical data 210 can be used to further train and improve the model and its output. Further as new data is added to the historical data 210, the translation module 232 can facilitate the organization and translation of the new data into a uniform set of terminology so the new data can support and expand the ability of the model 230 to recommend successful paraffin inhibitors for crude oils.

The translation module 232 may organize each data set so that the translated terminology is saved with and tagged as not being part of the original data set. When translated, the tags may appear as highlighted portions of the data, hyperlinked portions of the data that are linked to the original presentation of the data, foot-noted data, or the tags may be hidden and only revealed upon request of the user. The tags may also indicate a confidence level for the translation or how the data was collated with other data sets.

The extrapolation module 234 of the model 230 may extrapolate the historical data 210 to populate unknown properties and information in the historical data 210. The extrapolation module 234 may use the historical data 210 collated by the translation module 232 to populate unknown properties and information in the historical data 210 by borrowing and extrapolating known data from data sets related together by the translation module 232 to fill in unknown information in other related data sets. The confidence level assigned by the translation module 232 may be used by the extrapolation module 234 to modify the confidence level that the extrapolation module 234 initially assigns the extrapolated data inserted into an existing original data set.

The extrapolation module 234 may also extrapolate the performance data 216 if the cold finger test parameters and test procedures did not meet the thresholds set by a user in order to extrapolate the results that may be expected if the cold finger test was conducted in accordance with a standardized set of testing parameters. In some embodiments, the more extrapolation needed to be done by the extrapolation module 234 to adjust a cold finger test in the performance data 216, the lower the confidence level for the extrapolated data will be.

The extrapolation module 234 may also use new data input into the historical data 210 to further refine its extrapolations and may periodically update the extrapolated data based on new input data. This process over time may further refine the model and allow better correlations to be developed in the extrapolation module 234.

The extrapolation module 234 may also use the field information 218 to adjust the confidence level for related performance data 216, crude oil properties 214, and paraffin inhibitor properties 212. For example, a customer report that corroborates the effectiveness of a paraffin inhibitor use with a target crude oil, will increase the confidence level assigned to the associated data in historical data 210. Conversely, a published study may contradict a data set for crude oil properties that lowers the confidence level for the data set and associated data sets.

Consequently, the extrapolation module 234 allows the model 230 to periodically update and refine the associations, collations, and extrapolated data within the historical data 210. As the historical data 210 is refined, the model 230 may improve its accuracy in providing recommendations for which paraffin inhibitors the model 230 recommends to be used with a specific crude oil. Further, the model may be helpful in identifying correlations in the data to determine what properties of a crude oil should be principally considered in determine what makes a paraffin inhibitor effective in preventing the deposition of paraffins in equipment.

Further, the extrapolation module 234 may use the input data 250 and the historical data 210 to extrapolate the information in input data 250 to fill in any information missing from the input data 250. By extrapolating the input data, the model may be better able to identify information in the historical data 210 that may be relevant to the input data 250.

In some embodiments, the prediction module 236 may be used to predict what a property may be when the extrapolation module 234 may not be able to extrapolate an unknown property of performance data 216, crude oil properties 214, or paraffin inhibitor properties 212. For example, the prediction module 236 of the model 230 may use the historical data 210 to predict a paraffin inhibition efficiency of a paraffin inhibitor at a particular dosage with a target crude oil when actual cold finger test data is not available or if the available cold finger test data was conducted under non-standardized parameters. In some embodiments, the prediction module 236 may request extrapolated data sets from the extrapolation module 234 and the historical data 210 to predict a paraffin inhibition efficiency of a paraffin inhibitor at a particular dosage with a target crude oil. The prediction module 236 may update its internal module as actual test data is input into the historical data 210 and the prediction module 236 compares its prediction with the actual test results. Using the process, the prediction module 236 may also identify correlations between the paraffin inhibitor properties and crude oil properties and associate these correlations with an updated confidence interval as new data is added to the historical data 210.

The prediction module 236 may use different methodologies to predict a paraffin inhibition efficiency of a paraffin inhibitor at a particular dosage with a target crude oil. For example, the prediction module 236 may use machine learning techniques applied to generate parameters using methods such as a k-nearest neighbors algorithm, a Bayesian network algorithm, a random forest algorithm, a multiple layer decision tree, or other suitable and known methods.

The prediction module 236 may also output the reasons for the prediction of paraffin inhibition efficiency. These reasons may include existing cold finger test data in the historical data 210 for a combination of a target crude oil and a specific paraffin inhibitor, the confidence level determined by the model 230, and the similarities of crude oil properties, paraffin inhibitor properties, performance data, and field information related to a combination of a target crude oil and a specific paraffin inhibitor.

The confidence module 238 may be used to track and calculate a confidence level of data in the historical data 210 including the translated data provided by the translation module 232, the extrapolated data provided by the extrapolation module 234, and the predicted data provided by the prediction module 236. The confidence module 238 may also calculate the confidence level of data output by the module and publish the data and reasoning used as the basis for the confidence level.

A confidence level determined by the confidence module 238 may be provided as a color code, such as green, yellow, and red or variations thereof, or may be provided as a numerical score or statistical confidence interval. The confidence level determined by the confidence module 238 may be used to only publish outputs that exceed a predetermined confidence level.

The selection module 240 may select the output data 260 selected by the model 230 that meets a predetermined set of criteria, such as confidence interval or paraffin inhibition efficiency. The selection module 240 may organize the output data 260. For example, the selection model 240 may organize the output data 260 with the highest paraffin inhibition efficiency first. In some examples, the selection model 240 may organize the output data 260 in any other manner, such as by price, availability, location of a stockpile of the paraffin inhibitor, amount of paraffin inhibitor on site, and so forth.

Other modules 244 may also be used by the model 230. For example, a mapping module (not shown) may be used in conjunction with the translation module 232 to select standardized terminology to describe the location of a source of a crude oil. The mapping module may use the source location data and geological information of a crude oil to collate data sets that may share or be near the source location and may be from the same geological formation. The mapping module on the geographic limits of different crude oil formations that can be used by the system 200 or model 230 to group together or collate the historical data 210. Using the associated performance data, the crude oils properties 214, and paraffin inhibitors properties 212 may be similar enough to allow the model to make high confidence level recommendations of paraffin inhibitors that will work effectively with the crude oil produced from that crude oil formation because of a larger available data set.

Other modules 244 may also include a communication module (not shown) allowing the model 230 to communicate with historical data 210 that may be stored in the cloud, remote servers, or other remote computing devices. A communication module may also be used to allow remote access by users of the model 230. Additional modules may include a search module that searches for data that may be used to update the historical data 210.

Input data 250 may be provided to the model 230 including select target crude oil properties 252, inhibitor properties 254, use conditions 255, and user conditions 256. A user may want specific information about a paraffin inhibitor or type of paraffin inhibitor from the model 230 and so may input select inhibitor properties 254. The information that a user may provide for the select inhibitor properties 254 may include the name of a specific paraffin inhibitor, its composition, the chemical structure of specific molecules used in the paraffin inhibitor, such as chain length, length of branches, etc. Further, the select inhibitor properties 254 may limit output to paraffin inhibitors above or below a threshold related but not limited to a paraffin inhibition efficiency, price, dosage, or availability.

The target crude oil properties 252 may include the name or other identifier of the target crude oil, a description of the source location, such as a geographic description, address, global positioning system coordinates, sections in a government survey system, the name or description of the geological formation that the target crude oil may be sourced from, and other geographically related properties of the target crude oil properties 252. The target crude properties 252 may include API gravity, cloud point, pour point, wax appearance temperature, and composition data like SARA fractions, ratio of normal paraffins to branched plus cyclic paraffins, and/or weight percent Cx+ hydrocarbons as an indicator of paraffin content, where x is an integer parameter that can be selected depending on the needs of particular instances. Generally, x is above 15, such as 17 or 18, and can be as high as 20 in some cases. Crude oil properties may also include weight percent Cy+ hydrocarbons, where y is a different integer from x.

Use conditions 255 may also be described by a user. Use conditions 255 include but are not limited to environmental and field conditions. For example, use conditions 255 may include crude oil bulk temperature, pipeline outer wall temperature, weather conditions, flow rates, and pipeline and equipment materials and associated surface friction. Use conditions 255 may also include the thermal flux across a pipeline outer wall.

The user conditions 256 may specify the output data 260 that the user wants from the model. For example, the user may only want data, extrapolations, and predictions that have a confidence level above a certain threshold. The user may also specify how the recommendations should be ordered; alphabetically, by confidence level, by paraffin inhibition efficiency, by similarity to the target crude oil, etc.

The model 230 may use an artificial intelligence model or a machine learning model to correlate the input data 250, such as the target crude oil properties 252 with crude oil properties 214 of the historical data 210. In one embodiment, the model 230 may select a numerical tolerance to identify crude oils in the historical data 210 that may be similar to the target crude oil properties. For example, the model 230 may provide a 1 percent tolerance for weight percent for paraffins in a crude oil in seeking to identify similar crude oils in the historical data 210. Further, the model 230 may select a second or more properties to apply a numerical tolerance to for the identification of similar crude oils. These parameters may facilitate the identification of data that may be used to identify paraffin inhibitors having a high predicted paraffin inhibitor efficiency with the target crude oil.

The model 230 may then use the correlated crude oil properties 214 to identify performance data 216, paraffin inhibitor properties 212, field information 218, and other information 219 within the historical data 210. The model 230 may organize and output similar crude oil properties 262, recommended paraffin inhibitors 264 and their properties 266, the associated performance data 268, and other information 270 that may have been deemed relevant by the model 230 or requested by a user. As previously discussed above, the model 230 may extrapolate historical data 210 to replace unknown data in a data set of the historical data 210, and/or predict unknown information not available in the historical data 210 to output data including one or more of a list of crude oils having one or more properties within a numerical tolerance of the properties of the target crude oil, a list of paraffin inhibitors for use with the target crude oil; and a list of inhibitor properties whose inhibitor effectiveness is above a threshold.

The output data 260 may potentially include a custom paraffin inhibitor not based on a currently available inhibitor. For example, the model 230 may recommend mixing 2 or more known paraffin inhibitors at a specific ratio or percentage in order to achieve a superior paraffin inhibitor efficiency. Alternatively, the output data 260 may include a molecular structure for a custom paraffin inhibitor based on the correlations identified by the model 230.

The output data 260 may provide the inhibitor properties whose paraffin inhibitor effectiveness is above a numerical threshold, which may be provided by the user or predetermined. The model may also use parameter importance established during model building, where the parameter importance indicates dependence of inhibitor effectiveness or efficiency on the parameter, and the model may define a parameter importance score based on the parameter importance. Parameters may refer to the properties of the paraffin inhibitors, crude oils, and performance data that the model 230 correlates with a predicted high paraffin inhibitor efficiency for a particular crude oil.

Further, the model 230 may perform response surface modeling or other sensitivity analysis to determine variables of inhibitor structure to which inhibition effectiveness is most effective for a target crude oil. Additionally, or alternately, parameter importance to inhibitor effectiveness can be identified during model building and used in design or procurement of paraffin inhibitors. Such information can be used to optimize the molecular structure of a paraffin inhibitor for use with a specific target crude oil.

The model 230 may also base its recommendations on parameters such as market data available in the field information 218. For example, the model 230 may out a recommendation that notes the cost of precursors, or manufacturing methods and or equipment for a particular paraffin inhibitor may be unusually expensive or difficult to obtain or use so that implementation would be cost prohibitive. The target crude oil properties 252 may be input to include a cost threshold that may instruct the model 230 to remove potential paraffin inhibitors from the output if the cost to produce or the precursors are sold at a price high enough to make the use of that paraffin inhibitor cost prohibitive in spite of having a high paraffin inhibiting effectiveness and confidence level. Similarly, the dosage of a paraffin inhibitor may be used to increase or decrease the overall confidence level or placement of the paraffin inhibitor in the listing of recommendations if the dosage is too high to be economically feasible.

The model 230 may limit recommendations to off-the-shelf paraffin inhibitors that are currently stocked in sufficient quantities by commercial entities that regularly upload their inventory data to the historical data 210. In other cases, the model 230 may output crude oils similar to the target crude oil, based on a numerical score computed from numerically described properties of the input crude oil, along with all inhibitors tested with that crude oil in the historical data 210, and their test results. The model 230 may be configured to output a paraffin inhibitor functionality type predicted to be most effective for use with the target crude oil.

The translation module 232 of the model 230 may translate the output data and the describe the basis of the output data into plain English or another language. The use of explanatory text may prevent misunderstandings regarding the output data 260 and any recommendations made by the model 230.

Once the model 230 has output data 260, a user may test one or more of the paraffin inhibitor recommendations with the target crude oil 280 to determine its actual paraffin inhibition efficiency. Alternatively, the model 230 may also specify testing parameters as part of the output data to validate the model's extrapolations and predictions. The resulting test data may be used to make a final selection of the paraffin inhibitor and associated dosage 282. Further, the resulting test data may be input into the historical data 210. The new data may be used to recalibrate and re-train the model 230. The historical data 210 may also be used to update the model 230 on a regular periodic basis or only when a certain threshold of new data or certain types of new data are added to the historical data 210. For example, the model 230 may be recalibrated and re-trained when test data for previously recommended paraffin inhibitors for a target crude oil 280 are input into the historical data 210 or when the final selection of a paraffin inhibitor and its associated dosage for a target crude oil is made 282 and this information is input into the historical data 210. Alternatively, the model 230 may be recalibrated and re-trained when a report of observations of the actual usage of the paraffin inhibitor at the recommended dosage in a target crude oil within equipment is input into the historical data.

To calibrate the model 230, a calibration module 242 may tune the relationships between parameters of the historical data 210 and the model 230 until the output data are consistent with test results from recommended paraffin inhibitor tests with a target crude oil. In some embodiments, the parameters of the historical data 210 and the model 230 may be calibrated to minimize the differences between the extrapolated and predicted values versus the actual test results.

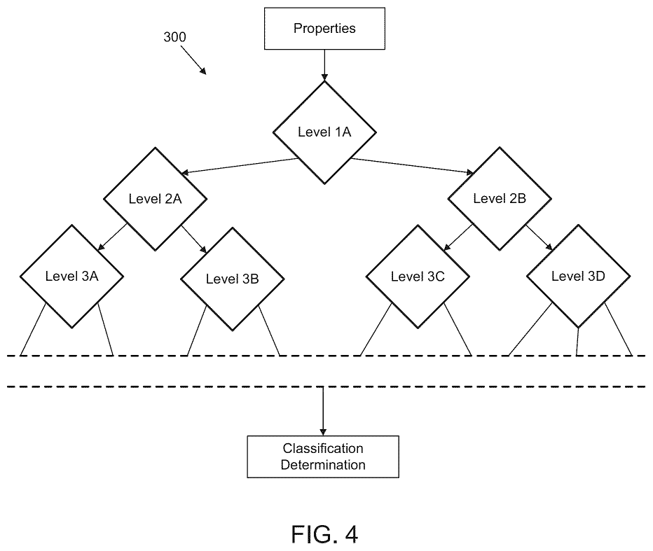

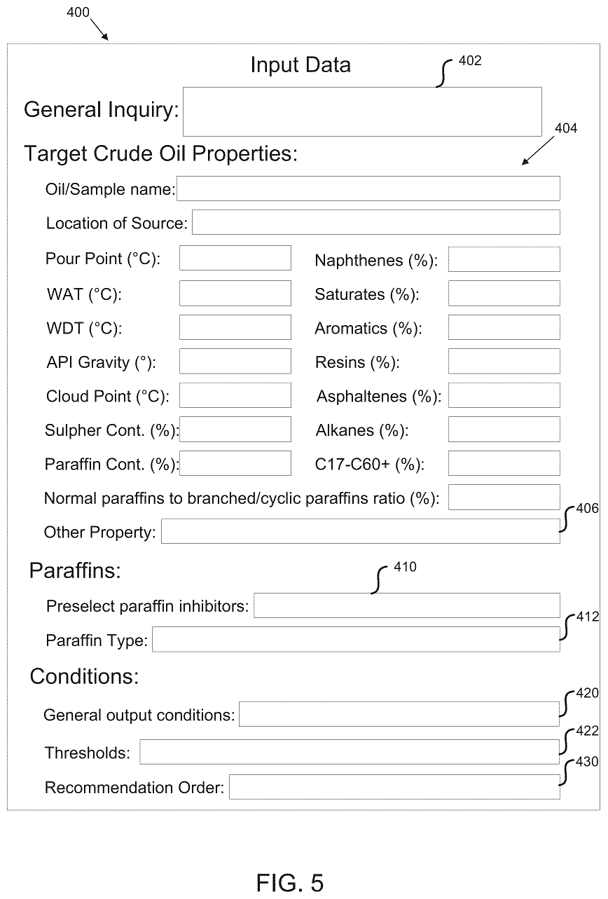

FIG. 4 illustrates a schematic of a decision tree 300 that may be implemented in the model 230 according to one embodiment. The decision tree 300 may be used by the model 230 to identify a paraffin inhibitor that may have a high paraffin inhibitor efficiency when used with a target crude oil. The decision tree 300 may be constructed based on decision rules determined by the model 230, using for example one or more machine learning techniques to identify rules that effectively classify the target crude oil. Alternately, some or all of the rules can be provided by a user or developer.

As shown, crude oil properties 302 may be input into the decision tree 300 and three or more classification tiers may be used to classify the crude oil. The model 230 may use the decision tree 300 to classify the crude oils and their properties in the historical data 210 as a means of organizing and collating the historical data 210. Additionally, the model 230 may use the decision tree 300 to classify a target crude oil and its properties. Each classification layer may be used separate crude oils by a specific property.

For example, the first classification made on level 1A 302 may be based the crude oil's American Petroleum Institute (API) gravity. Each level of the decision tree 300 may use the same property for classification or different properties based on the classification made in a prior level. Level 2A and 2B may classify the crude oil based on weight percent of paraffins while Level 3A, 3B, 3C, and 3D may classify the crude oil based on its normal paraffins to branched or cyclic paraffin ratio. Alternatively, Level 2A may classify a crude oil based on the percent of saturates in the crude oil, while 2B classifies the crude oil on the percent of resins. Level 3A may classify the crude oil based on the wax appearance temperature of the crude oil. Level 3B may classify the crude oil based on the pour point of the crude oil. Level 3C may classify the crude oil on the percent of asphaltenes in the crude oil. Level 3D may classify the crude oil based on its normal paraffins to branched or cyclic paraffin ratio.

Alternatively, the model 230 may use the decision tree 300 to classify cold finger test results in the historical data 210. For example, Level 1A may classify the properties of the cold finger test by the crude oil's American Petroleum Institute (API) gravity. Level 2A may classify the cold finger test by the name of the paraffin inhibitor used. Level 2B may classify the cold finger test by the paraffin content of the crude oil. Levels 3A, 3B, and 3C may classify the cold finger test by the finger temperature used while Level 3D may classify the cold finger test by chain length of the principal polymer in the paraffin inhibitor. A level may have more than two options to choose from. As shown, Level 3D may have 3 classification choices. For example, the 3 choices may represent ranges of a property, numerical thresholds, or levels of poor, good, and excellent.

As cold finger tests are classified, the paraffin inhibitors may eventually be grouped as having excellent, good, and poor general paraffin inhibition efficiency or may be grouped according to specific paraffin inhibition efficiency related to a specific crude oil. Further, the decision tree 300 may be used to help identify paraffin recommendations from historical data 210 and assist the model in quickly outputting paraffin recommendations. A confidence level may be associated with the paraffin recommendations for a specific crude oil and output with the recommendations or used to organize the recommendations.

FIG. 5 is a user interface 400 for the model 230 according to one embodiment. The user interface 400 may be customized to a user's preferences. In some embodiments, the user interface 400 may display a general inquiry 402 that accepts plain language requests. The plain language requests maybe interpreted by the translation module 232 and then processed by the model 230.

The user interface 400 may also present a series of options to facilitate the input of data. As shown, target crude oil properties 404 may be input based on what a user may know and may include information in the other properties section 406 for information of the crude oil not already listed. Further, one or more specific paraffin inhibitors 410 or types of paraffin inhibitors 412 may be input. A user may also specify general output conditions 420 using plain language. For example, a user may request in the general output conditions 420 that all similar crude oils be output with the most similar first together with associated paraffin inhibitor test data and limit the results to the first 20 similar crude oils. Thresholds 422 may also be set out so that the recommendations only provide data that meet the thresholds 422. A user may also specify the final recommendation order 430, such as by paraffin inhibitor efficiency, grouped by type of paraffin inhibitor, or most analogous crude oils and associated paraffin inhibitors having the highest paraffin inhibitor efficiency first.

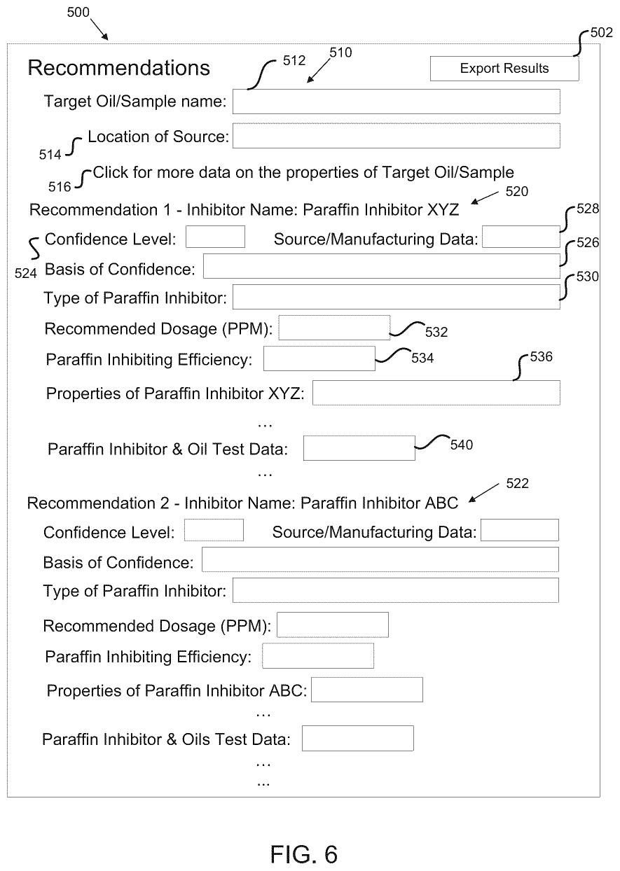

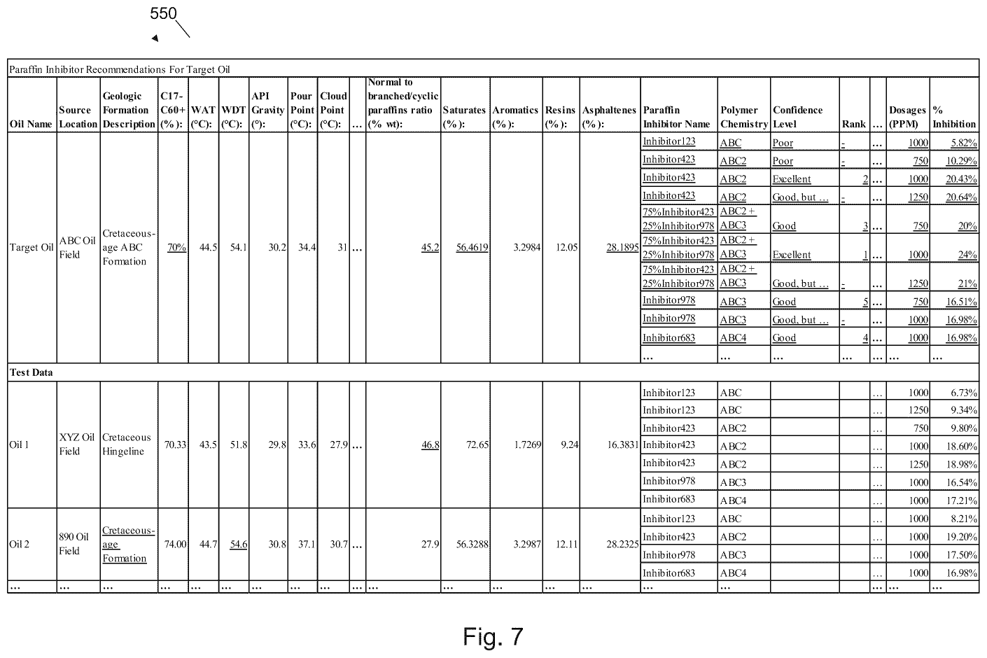

FIG. 6 shows a user interface 500 displaying an output of the model 230. As shown, the user interface 500 is displaying its recommendations for paraffin inhibitors for use with a target crude oil. The user interface 500 may display an option to export the raw data for the user to see what data is original and what has been extrapolated or predicted by the model 230. The raw data may be exported in different formats including but not limited to a spreadsheet as shown in FIG. 7.

The user interface 500 may display the properties of the target crude oil 510 including its name 512 and location of the source 514 of the target crude oil. Optionally, a link may be included for all of the property information of the target crude oil may be accessed. The information may be presented as a list or as a spreadsheet.

The user interface 500 may display the properties of the recommended paraffin inhibitors 520 and 522. As shown, a variety of properties of the paraffin inhibitors may be displayed. The confidence level 524 may be calculated by the model 230 from various parameters and may be presented in a numerical manner, a level displayed as a color such as red, yellow, and green, an indicator such as poor, good, and excellent and such other ways of indicating a confidence level. The confidence level may be based on the amount of data available, the number of confirming data sets, and the number of contradictory data sets. The confidence level may also be determined based on the confidence factor of the underlying tests and studies. Further the confidence level may also be determined based on the extrapolation and prediction analysis of the model 230.

When published in the output data, the user interface 500 may display the key aspects that form basis of the confidence level 526. For example, the basis of the confidence level 526 may state “This recommendation is based on an existing cold finger test for this paraffin inhibitor and the target crude oil.” The basis of the confidence level 526 may also state “This paraffin inhibitor and the target crude oil are highly ranked by the model based on the subject matter expert designed decision tree.” Additionally, the basis of the confidence level 526 may also state the XXX (Bayesian Network, Random Forest) algorithm predicts a paraffin inhibitor efficiency of 80% with a confidence level of 0.92.″

Alternatively, the basis of the confidence level 526 may state “There is very little data available for this combination of crude oil and paraffin inhibitor” and provide a low confidence level for the recommendation. Similarly, the basis of the confidence level 526 may state “This data set has a high level of extrapolation. The confidence level in these predictions have a confidence level of yellow. Please test the recommended paraffin inhibitor with the target crude oil before using in a commercial application.”

The user interface 500 may display source and/or manufacturing data 528 for the recommended paraffin inhibitor. This section of the user interface 500 may also include pricing and availability information. In the event that the model 230 has recommended a paraffin inhibitor created by mixing two know paraffin inhibitors, the proportions of each component may be provided in the source and/or manufacturing data 528 section of the user interface 500.

The user interface 500 may display the type of paraffin inhibitor 530, the recommended dosage 532, and the paraffin inhibitor efficiency 534 for the target crude oil. Further, other properties of the recommended paraffin inhibitor 536 such as polymer chemistry, a listing of components, and other properties may be displayed by the user interface 500 or in the exported results 502.

Additionally, the user interface may display the recommended paraffin inhibitor and oil test data 540. The test data may be ordered with the most relevant test data and crude oil specification listed first.

FIG. 7 is an example of exported results 502 shown in a spreadsheet 550 from the user interface 500. As shown, properties for the target crude oil and similar crude oils are listed. The underlined values are data that has been extrapolated or predicted by the model 230. Alternatively, extrapolated or predicted values may be noted with a * or may be highlighted in the spreadsheet. Extrapolated or predicted values may also be hyperlinked to the original data that the extrapolation or prediction was based upon. In this example, the model 230 has provided confidence levels as Poor, Good, but . . . , Good, and Excellent. The Good, but . . . confidence level was applied to secondary dosages where more effective dosages of the same paraffin inhibitor were recommended. The paraffin inhibitors have been ranked with paraffin inhibitors having a confidence level below Good, not receiving a recommendation ranking.

As shown, the spreadsheet 550 shows a list of crude oils having one or more properties. In this case, the target crude oil may not have any related data in the historical data 210. Consequently, the model 230 may have searched the historical data 210 to identify similar crude oil information. In this example, the model 230 selected crude oils that have an American Petroleum Institute (API) gravity within +/−1° of the API gravity of the target crude oil. Secondarily, the model 230 may select crude oils that have a weight percent of C17—C60+ hydrocarbons within +/−5 percent of the weight percent of C17-C60+ hydrocarbons of the target crude oil. Consequently, Oil 1 and Oil 2 are within a numerical tolerance of the properties of the target crude oil.

The model 230 in this example identified Oil 1 and Oil 2 and then extrapolated the performance data for the target crude oil from Oil 1 and Oil 2. Based on these extrapolations, the model 230 has predicted the listed paraffin inhibitor efficiencies. The model 230 then output the list of paraffin inhibitors for use with the target crude oil. As shown, a list of inhibitor properties ordered for use with the target crude oil based on the predicted paraffin inhibitor efficiency value, with additional dosages of the same paraffin inhibitor being not selected.

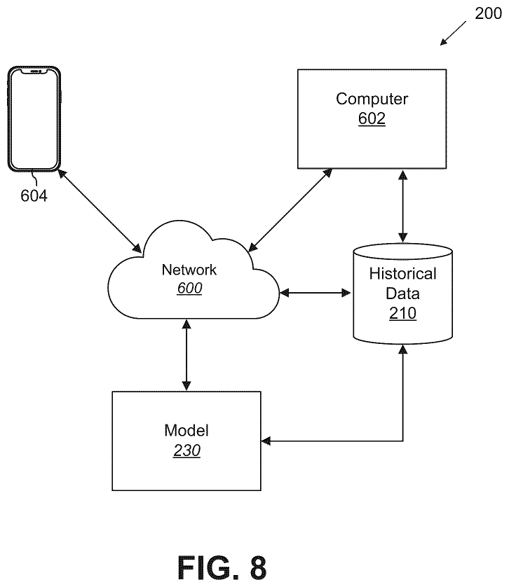

FIG. 8 is a schematic representation of the system 200, according to at least one embodiment of the present disclosure. The system 200 includes a model 230. The model 230 may periodically undergo calibration and machine learning using historical data 210 and any new updates to the historical data 210 that may be input by users via an interface, such as a computer 602 or mobile device 604. The model 230 and historical data 210 may be maintained on the network 600 such that extrapolations, estimations, predictions, and calibrations are implemented on the network 600. The network 600 may be a cloud server or a server in a data center.

Furthermore, the components of the system 200 may, for example, be implemented as one or more operating systems, as one or more stand-alone applications, as one or more modules of an application, as one or more plug-ins, as one or more library functions or functions that may be called by other applications, and/or as a cloud-computing model. Thus, the components may be implemented as a stand-alone application, such as a desktop or mobile application. Furthermore, the components may be implemented as one or more web-based applications hosted on a remote server. The components may also be implemented in a suite of mobile device applications or “apps.”

The model 230 may be operationally coupled to an interface unit such as a computer 602 to simplify using the model 230. The computer 602 may provide users with an uncomplicated way to input information about a target crude oil to be used to query the model 230, and to view information output by the model 230. The model output may take the form of a set of recommended inhibitors, optionally along with predicted numerical paraffin inhibition efficiencies, such as PIE. The list may be sorted automatically or by user selection. Alternately, or additionally, the model output can take the form of a list of crude oils having one or more characteristics that are within a numerical tolerance of characteristics of the target crude oil. For example, the user can provide a numerical tolerance for API gravity and weight percent C17+ hydrocarbons and the model can return all crude oils in the database within those numerical tolerances of the target crude oil, along with inhibitor performance data for those crude oils. Alternately or additionally, the model output can take the form of a set of inhibitor characteristics predicted to have the largest effect on inhibitor performance for the target crude oil. The user can, optionally, supply a numerical tolerance, and the model can identify inhibitor characteristics, such a molecular structure characteristics or inhibitor type, predicted to have larger effect on inhibition performance than the provided tolerance.

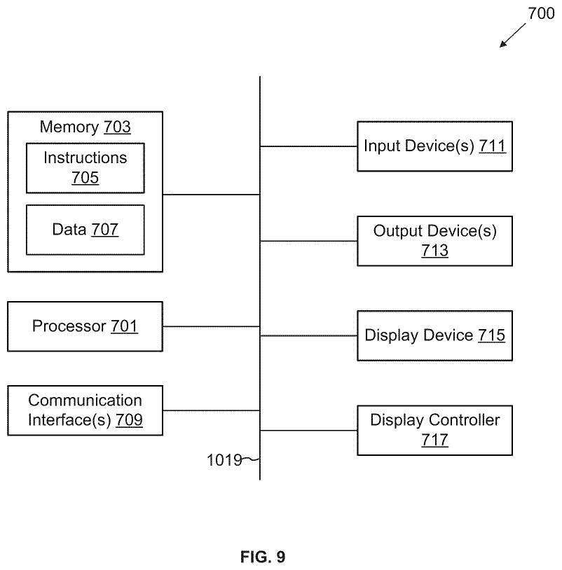

FIG. 9 illustrates certain components that may be included within a computer system 700. One or more computer systems 700 may be used to implement the system 200 described herein.

The computer system 700 includes a processor 701. The processor 701 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 701 may be referred to as a central processing unit (CPU). Although just a single processor 701 is shown in the computer system 700 of FIG. 9, in an alternative configuration, a combination of processors (e.g., an ARM and DSP) could be used.

The computer system 700 also includes memory 703 in electronic communication with the processor 701. The memory 703 may be any electronic component capable of storing electronic information. For example, the memory 703 may be embodied as random access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 705 and data 707 may be stored in the memory 703. The instructions 705 may be executable by the processor 701 to implement some or all of the functionality disclosed herein. Executing the instructions 705 may involve the use of the data 707 that is stored in the memory 703. Any of the various examples of modules and components described herein may be implemented, partially or wholly, as instructions 705 stored in memory 703 and executed by the processor 701. Any of the various examples of data described herein may be among the data 707 that is stored in memory 703 and used during execution of the instructions 705 by the processor 701.

A computer system 700 may also include one or more communication interfaces 709 for communicating with other electronic devices. The communication interface(s) 709 may be based on wired communication technology, wireless communication technology, or both. Some examples of communication interfaces 709 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 700 may also include one or more input devices 711 and one or more output devices 713. Some examples of input devices 711 include a keyboard, mouse, microphone, remote control device, button, joystick, trackball, touchpad, and lightpen. Some examples of output devices 713 include a speaker and a printer. One specific type of output device that is typically included in a computer system 700 is a display device 715. Display devices 715 used with embodiments disclosed herein may utilize any suitable image projection technology, such as liquid crystal display (LCD), light-emitting diode (LED), gas plasma, electroluminescence, or the like. A display controller 717 may also be provided, for converting data 707 stored in the memory 703 into text, graphics, and/or moving images (as appropriate) shown on the display device 715.

The various components of the computer system 700 may be coupled together by one or more buses, which may include a power bus, a control signal bus, a status signal bus, a data bus, etc. For the sake of clarity, the various buses are illustrated in FIG. 9 as a bus system 719.

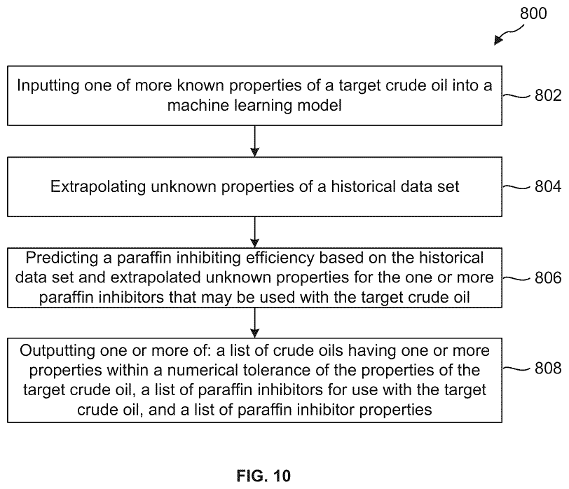

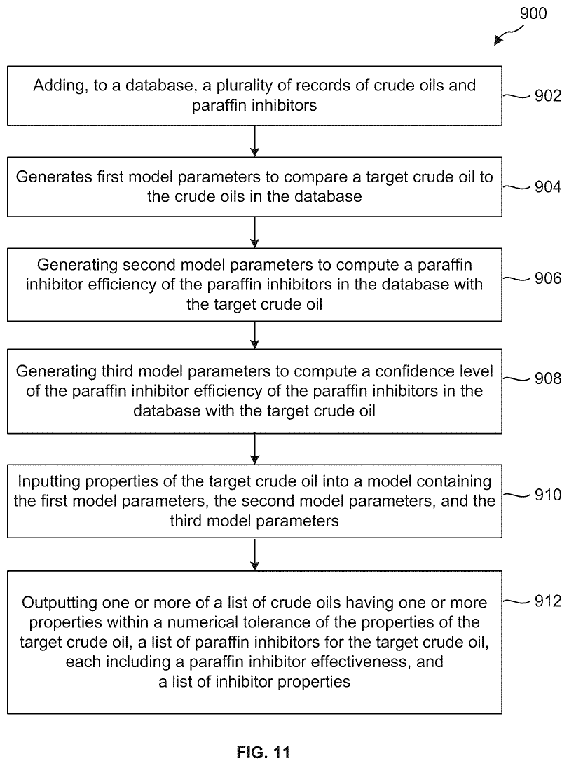

FIG. 10 through FIG. 12, the corresponding text, and the examples provide a number of different methods, systems, devices, and computer-readable media of the system 200. In addition to the foregoing, one or more embodiments can also be described in terms of flowcharts comprising acts for accomplishing a particular result, as shown in FIG. 10 through FIG. 12, may be performed with more or fewer acts. Further, the acts may be performed in differing orders. Additionally, the acts described herein may be repeated or performed in parallel with one another or parallel with different instances of the same or similar acts.

FIG. 10 is a flow chart of a method for selecting paraffin inhibitors for a target crude oil. A user inputs one of more known properties of a target crude oil into a machine learning model at 802. The machine learning model extrapolates unknown properties of a historical data set at 804. The machine learning model may be trained on the historical data set. The historical data set includes one or more properties of a plurality of crude oils, one or more properties of a plurality of paraffin inhibitors, and one or more paraffin inhibiting efficiencies of the paraffin inhibitors with the crude oils. The machine learning model may predict a paraffin inhibiting efficiency based on the historical data set and extrapolated unknown properties for the one or more paraffin inhibitors that may be used with the target crude oil at 806. The machine learning model may output one or more of a list of crude oils having one or more properties within a numerical tolerance of the properties of the target crude oil, a list of paraffin inhibitors for use with the target crude oil; and a list of paraffin inhibitor properties at 808.

The user or the machine learning model may select or design a chosen paraffin inhibitor, based on the output, for use with the target crude oil. In one embodiment, the user may test a plurality of the output paraffin inhibitors with the target crude oil. Based on the actual test data, the user selects the paraffin inhibitor to be used with the target crude oil. Once selected, a user may validate the predicted paraffin inhibiting efficiency with testing on the paraffin inhibitor and the target crude oil and add the results of the actual testing to the historical data set. The historical data set may include a cold finger test results of a combination of a crude oil and a paraffin inhibitor.

The one or more properties of a plurality of crude oils may include a location of a source of one of the plurality of crude oils. The one or more properties of a plurality of crude oils may also include one or more of a location of a source of one of the plurality of crude oils, carbon chain distribution of the paraffins, or the wax appearance temperature (WAT). The one or more properties of a plurality of paraffin inhibitors may include one or more of polymer length, side chain length, or the ratio between polymer length and side chain length.

In some embodiments, the machine learning model may design a paraffin inhibitor and include the newly designed paraffin inhibitor as an output. Without actual test data for the newly designed paraffin, a user will test the newly designed paraffin inhibitor with the target crude oil. Once tested, a user will add the results of the actual testing to the historical data set. This data set for the newly designed paraffin may be used to calibrate the machine learning model.

As part of the method, the machine learning model may calculate a paraffin inhibitor efficiency value for each combination of a paraffin inhibitor and the target crude oil using the historical data set. Prior to the outputting step, the machine learning model may order the paraffin inhibitors for use with the target crude oil based on the predicted paraffin inhibitor efficiency value. As used herein, the term “ordering” means to arrange in a methodical way and should be interpreted to include the ranking of paraffin inhibitors by paraffin inhibitor efficiency. For example, ordering the paraffin inhibitors may mean placing the most efficient paraffin inhibitor at the top of an outputted list. Alternatively, ordering the paraffin inhibitors may mean merely indicating a set of paraffin inhibitors have good efficiency compared to other paraffin inhibitors that have poor efficiency with a target crude oil.

The machine learning model may calculate a confidence level for the paraffin inhibitor efficiency using a confidence indicator based on one or more of a decision tree classifying one or more properties of a plurality of crude oils, one or more properties of a plurality of paraffin inhibitors, or a cold finger test result in the historical data set. The machine learning model may use the confidence level to limit the output to one or more crude oils, paraffin inhibitors, and inhibitor properties that have a confidence level above a predetermined threshold.

The machine learning model in predicting the paraffin inhibiting efficiency may use a Bayesian Network, Random Forest, to relate the paraffin inhibitor efficiency to the range of values representing the crude oil properties and paraffin inhibitor structural data.

FIG. 11 is a flow chart of another method 900. A user or a machine learning model adds, to a database, a plurality of records of crude oils and paraffin inhibitors at 902. Each record includes one or more of properties of the crude oil including identification of a source of a crude oil, paraffin content, carbon chain length distribution of paraffins, wax appearance temperature, American Petroleum Institute (API) gravity, cloud point, pour point, and a ratio of normal paraffins to branched plus cyclic paraffins, and saturates, aromatics, resins, and asphaltenes (SARA) fractions, identification of a paraffin inhibitor, type of the paraffin inhibitor, molecular properties of the paraffin inhibitor, dosage of the paraffin inhibitor in the crude oil and paraffin inhibition results of the paraffin inhibitor in the crude oil. The machine learning model generates first model parameters to compare a target crude oil to the crude oils in the database at 904. The machine learning model generates second model parameters to compute a paraffin inhibitor efficiency of the paraffin inhibitors in the database with the target crude oil at 906 and generates third model parameters to compute a confidence level of the paraffin inhibitor efficiency of the paraffin inhibitors in the database with the target crude oil at 908. The machine learning model inputs properties of the target crude oil into a model containing the first model parameters, the second model parameters, and the third model parameters at 910. The machine learning model then output one or more of a list of crude oils having one or more properties within a numerical tolerance of the properties of the target crude oil, a list of paraffin inhibitors for the target crude oil, each including a paraffin inhibitor efficiency, and a list of inhibitor properties at 912.

The machine learning model or a user may select or design a chosen paraffin inhibitor, based on the output, for use with the target crude oil. In one embodiment, the user may test a plurality of the output paraffin inhibitors with the target crude oil. Based on the actual test data, the user selects the paraffin inhibitor to be used with the target crude oil. Once selected, a user may validate the confidence level with testing on the paraffin inhibitor and the target crude oil and add the results of the actual testing to the historical data set. The historical data set may include a cold finger test results of a combination of a crude oil and a paraffin inhibitor.

The machine learning model may extrapolate unknown properties of each record using the database to generate one or more of the first model parameters, second model parameters, and third model parameters. The machine learning model may predict the paraffin inhibiting efficiency using a Bayesian network to relate the paraffin inhibitor efficiency to the range of values representing the crude oil properties and paraffin inhibitor structural data. The machine learning model may also correlate the paraffin inhibitor efficiency with a range of values representing the crude oil properties and paraffin inhibitor structural data.

In computing a confidence level for the paraffin inhibitor efficiency, a machine learning model may use a confidence indicator based on a decision tree classifying one or more properties of a plurality of crude oils, one or more properties of a plurality of paraffin inhibitors, or a cold finger test result in the database. The machine learning model may use the confidence level to limit the output to one or more crude oils, paraffin inhibitors, and inhibitor properties that have a confidence level above a predetermined threshold.

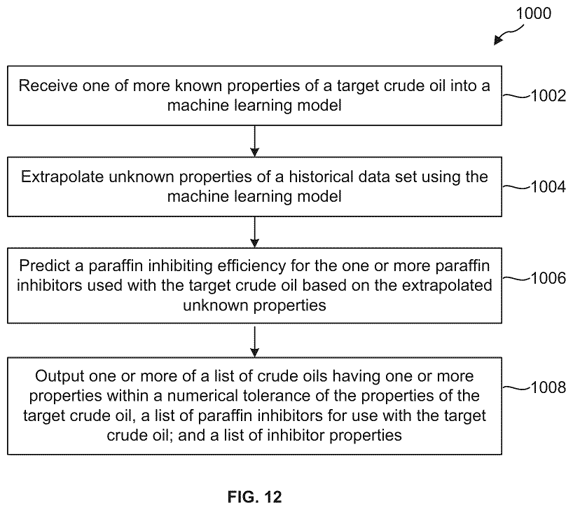

FIG. 12 is a flow chart of another method 1000. A computing system including a processor; and a memory including instructions, a database, and a machine learning model that may cause the processor to implement the method 1000. The computer system receives one of more known properties of a target crude oil into the machine learning model at 1002. The computer system the extrapolates unknown properties of a historical data set using the machine learning model at 1004. The machine learning model is trained on the historical data set including one or more properties of a plurality of crude oils, one or more properties of a plurality of paraffin inhibitors, and paraffin inhibiting effectiveness of the paraffin inhibitors with the crude oils. The computer system predicts a paraffin inhibiting efficiency for the one or more paraffin inhibitors used with the target crude oil based on the extrapolated unknown properties at 1006. The computer system then outputs one or more of a list of crude oils having one or more properties within a numerical tolerance of the properties of the target crude oil, a list of paraffin inhibitors for use with the target crude oil, and a list of inhibitor properties.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method for selecting paraffin inhibitors for a target crude oil, the method comprising:
inputting one of more known properties of a target crude oil into a machine learning model;
extrapolating unknown properties of a historical data set using a machine learning model, the machine learning model trained on the historical data set including one or more properties of a plurality of crude oils, one or more properties of a plurality of paraffin inhibitors, and one or more paraffin inhibiting efficiencies of the paraffin inhibitors with the plurality of crude oils;
predicting a paraffin inhibiting efficiency based on the historical data set and extrapolated unknown properties for the plurality of paraffin inhibitors that may be used with the target crude oil;
outputting one or more of:
a list of crude oils having one or more properties within a numerical tolerance of the properties of the target crude oil;
a list of paraffin inhibitors for use with the target crude oil; and
a list of paraffin inhibitor properties.

2. The method of claim 1, further comprising selecting or designing an output paraffin inhibitor, based on the output, for use with the target crude oil.

3. The method of claim 2, further comprising validating the predicted paraffin inhibiting efficiency by testing the paraffin inhibitor with the target crude oil; and adding results of the testing to the historical data set.

4. The method of claim 1, further comprising calculating a paraffin inhibitor efficiency value for each combination of a paraffin inhibitor and the target crude oil using the machine learning model and the historical data set.

5. The method of claim 4, further comprising ordering the paraffin inhibitors for use with the target crude oil based on the predicted paraffin inhibitor efficiency.

6. The method of claim 1, wherein the historical data set comprises a cold finger test result of a combination of a crude oil and a paraffin inhibitor.

7. The method of claim 6, further comprising calculating a confidence level for the paraffin inhibitor efficiency using a confidence indicator based on one or more of a decision tree classifying one or more properties of a plurality of crude oils, one or more properties of a plurality of paraffin inhibitors, or a cold finger test result in the historical data set.

8. The method of claim 7, further comprising using the confidence level to limit the output to one or more crude oils, paraffin inhibitors, and inhibitor properties that have a confidence level above a predetermined threshold.

9. The method of claim 1, wherein predicting the paraffin inhibiting efficiency, the machine learning model correlates the paraffin inhibitor efficiency with a range of values representing the crude oil properties and paraffin inhibitor structural data.

10. The method of claim 1, wherein the one or more properties of a plurality of crude oils includes a location of a source of one of the plurality of crude oils.

11. The method of claim 1, wherein the one or more properties of a plurality of crude oils comprises one or more of a location of a source of one of the plurality of crude oils, a carbon chain distribution of the paraffins, or a wax appearance temperature (WAT).

12. The method of claim 1, wherein the one or more properties of a plurality of paraffin inhibitors comprises one or more of a polymer length, a side chain length, or a ratio between polymer length and side chain length.

13. A method comprising:
adding, to a database, a plurality of records of crude oils and paraffin inhibitors, each record including one or more of:
properties of the crude oils including identification of a source of a crude oil, paraffin content, carbon chain length distribution of paraffins, wax appearance temperature, American Petroleum Institute (API) gravity, cloud point, pour point, and a ratio of normal paraffins to branched plus cyclic paraffins, and saturates, aromatics, resins, and asphaltenes (SARA) fractions;
identification of a paraffin inhibitor;
type of the paraffin inhibitor;
molecular properties of the paraffin inhibitor;
dosage of the paraffin inhibitor in the crude oil; and
paraffin inhibition results of the paraffin inhibitor in the crude oil;
generating first model parameters to compare a target crude oil to the crude oils in the database;
generating second model parameters to compute a paraffin inhibitor efficiency of the paraffin inhibitors in the database with the target crude oil;
generating third model parameters to compute a confidence level of the paraffin inhibitor efficiency of the paraffin inhibitors in the database with the target crude oil;
inputting properties of the target crude oil into a model containing the first model parameters, the second model parameters, and the third model parameters; and
outputting one or more of:
a list of crude oils having one or more properties within a numerical tolerance of the properties of the target crude oil;
a list of paraffin inhibitors for the target crude oil, each including a paraffin inhibitor efficiency; and
a list of inhibitor properties.

14. The method of claim 13, further comprising selecting or designing a chosen paraffin inhibitor, based on the output, for use with the target crude oil.

15. The method of claim 13, further comprising validating the confidence level with testing on the paraffin inhibitor and the target crude oil; and adding results of the testing to the database.

16. The method of claim 13, further comprising extrapolating unknown properties of each record using the database to generate one or more of the first model parameters, second model parameters, and third model parameters.

17. The method of claim 13, further comprising predicting the paraffin inhibiting efficiency using a Bayesian network to relate the paraffin inhibitor efficiency to a range of values representing the crude oil properties and paraffin inhibitor structural data.

18. The method of claim 13, wherein computing a confidence level for the paraffin inhibitor efficiency includes using a confidence indicator based on a decision tree classifying one or more properties of a plurality of crude oils, one or more properties of a plurality of paraffin inhibitors, or a cold finger test result in the database.

19. The method of claim 18, further comprising using the confidence level to limit the output to one or more crude oils, paraffin inhibitors, and inhibitor properties that have a confidence level above a predetermined threshold.

20. A computing system comprising:
a processor; and
a memory including instructions, a database, and a machine learning model that cause the processor to:
receive one of more known properties of a target crude oil into the machine learning model;
extrapolate unknown properties of a historical data set using the machine learning model, the machine learning model trained on the historical data set including one or more properties of a plurality of crude oils, one or more properties of a plurality of paraffin inhibitors, and paraffin inhibiting efficiencies of the paraffin inhibitors with the crude oils;
predict a paraffin inhibiting efficiency for the one or more paraffin inhibitors used with the target crude oil based on the extrapolated unknown properties;
output one or more of:
a list of crude oils having one or more properties within a numerical tolerance of the properties of the target crude oil;
a list of paraffin inhibitors for use with the target crude oil; and
a list of inhibitor properties.
